(19) United States
(12) Patent Application Publication
(10) Pub. No.: US 2017/0061828 A1
(43) Pub. Date: Mar. 2, 2017

(54) FUNCTIONAL PROSTHETIC DEVICE TRAINING USING AN IMPLICIT MOTOR CONTROL TRAINING SYSTEM

(71) Applicant: ARIZONA BOARD OF REGENTS ON BEHALF OF ARIZONA STATE UNIVERSITY, Tempe, AZ (US)

(72) Inventors: Panagiotis Artemiadis, Tempe, AZ (US); Mark Ison, Tempe, AZ (US)

(21) Appl. No.: 15/245,108

(22) Filed: Aug. 23, 2016

Related U.S. Application Data
(60) Provisional application No. 62/209,212, filed on Aug. 24, 2015.

(51) Int. Cl. G09B 23/30; A61B 34/30; A61B 5/0488; G09B 5/02

(52) U.S. Cl. CPC G09B 23/30; G09B 5/02; A61B 34/30; A61B 5/04888; A61B 2505/09

(57) ABSTRACT

An implicit motor control training system for functional prosthetic device training is provided as a novel approach to rehabilitation and functional prosthetic controls by taking advantage of a human's natural motor learning behavior while interacting with electromyography.

### CROSS REFERENCE TO RELATED APPLICATIONS

This is a non-provisional application that claims benefit to U.S. provisional application Ser. No. 62/209,212 filed on Aug. 24, 2015, which is herein incorporated by reference in its entirety.

### FIELD

The present disclosure generally relates to functional prosthetic device training and in particular to prosthetic device training using an implicit motor control training system.

### BACKGROUND

A variety of functional prostheses have been proposed using electromyography (EMG) for control, but have had limited success due to user concerns over functionality and reliability. Current state-of-the-art control schemes try to mimic a user's natural kinematics between muscle activity and joint movement, but this complex relationship is not easily modeled, and stochastic noise in EMG makes the controls increasingly unreliable over time.

There are approximately 185,000 amputation procedures in the United States each year. Prosthetic devices provide an opportunity for individuals with amputations to regain independent lifestyles. Myoelectric prosthetics, with control inputs representing residual muscle activity via electromyography (EMG), have potential for enhanced functionality compared to cosmetic and body-powered (i.e. cable-driven) prostheses. However, commercially available myoelectric prostheses have struggled to provide both functional and reliable controls, leading 75% of users to reject these prostheses in favor of less functional, more reliable options. Current state-of-the-art myoelectric prostheses attempt to mimic the natural kinematics between muscle activity and joint movements, with the assumption that an initial intuitive control is required for user acceptance. However, the complex kinematics results in computationally expensive control algorithms, which require simplification to reduce power consumption at the expense of functionality (i.e. no proportional/simultaneous controls, and/or less active degrees of freedom). In addition, the stochasticity of EMG makes the resulting control schemes unreliable over time, requiring consistent algorithm recalibration or user retraining. Moreover, there is no guarantee a user has voluntary control of the muscles necessary to reproduce natural kinematics. Recent studies have shown that users, when given time to become familiar with a given prosthetic device, can achieve similar performances regardless of the kinematic relationship, or initial intuitiveness, of the device's control scheme. However, the learning curve is inversely proportional to the device's initial intuitiveness.

Surface electromyography (EMG) has been investigated as a potential input to robotic controls for over half a century. Myoelectric interfaces utilize EMG for real-time, non-invasive access to muscle activity, which is ideal for enhancing many applications in human-machine interaction such as prostheses and robot teleoperation. However, the desire for user-friendly myoelectric applications controlling simultaneous multifunctional robotic devices has yet to be achieved in commercial applications. Simultaneous multifunctional control has often been proposed using pattern recognition techniques, such as artificial neural networks and support vector machines, to relate EMG inputs with desired outputs and ultimately predict a user's intent. This approach is limited by the functionality provided in the training set, and restricted by threats of performance degradation during actual use due to transient changes in EMG. Thus, real-time performance requires users to adjust to unpredictable responses for complex motions or restrict controls to those accurately predicted.

Other approaches propose fixed mappings with proportional controls, where humans learn to control the application by identifying the relationship between EMG inputs and control outputs. These studies often use EMG signals to control a cursor on a monitor. While interacting with the interface, healthy subjects consistently learn the mapping between input and output, and develop new synergies as they modify muscle activity to correspond with higher-level intent. Learning has been verified in both intuitive (e.g. outputs related to limb motions) and non-intuitive (e.g. random) mapping functions. Other studies have identified similar learning patterns using abstract mappings similar to cursor control to operate a prosthetic hand, and suggest that robotic control can be studied using cursor control paradigms.

### BRIEF DESCRIPTION OF THE DRAWINGS

FIG. 1 illustrates an experimental setup having a Delsys EMG system and either the robotic or visual interface;

FIGS. 2A and 2B are a pair of charts showing the mapping of input EMG amplitudes to three output control axes using a mapping function;

FIGS. 3A and 3B are a pair of pictures showing the prosthetic hand configuration in the testing phase with a normal configuration (FIG. 3A) and rotated configuration (FIG. 3B);

FIG. 4 is a chart showing the performance learning curve and retention;

FIG. 5 is a chart showing the control efficiency learning curve;

FIGS. 6A-6D illustrate a sequence for robot control tasks with the robotic hand in a fixed orientation; and

FIG. 7 is a chart showing explicit vs. implicit robot control performance test.

Corresponding reference characters indicate corresponding elements among the view of the drawings. The headings used in the figures do not limit the scope of the claims.

### DETAILED DESCRIPTION

As disclosed herein, an implicit motor control training system for (IMCTS) functional prosthetic device training is provided as a novel approach to rehabilitation and functional prosthetic controls by taking advantage of a human's natural motor learning behavior while interacting with EMG. It is suggested in this disclosure that humans can learn any arbitrary mapping between muscle activity (as recorded by EMG) and prosthetic function, thereby providing more reliable controls over time. The chief objective is to create an implicit motor control training system that helps users overcome an initial learning curve and intuitively control N (N>0) degrees of freedom on a prosthetic device simultaneously and proportionally using only M (M>N) muscles. The training system allows users to develop new muscle synergies associated with the non-intuitive control of the prosthetic device, such that after rehabilitation and training, the controls “feel” intuitive. In addition, a related control scheme provides enhanced functionality and reliability over current state-of-the-art methods, thereby addressing the two major limitations in commercial prostheses. Experimental results demonstrate the natural motor learning invoked by interaction with EMG, and confirm that a training system designed to develop and refine muscle synergies associated with a control scheme following the guidelines outlined in this disclosure lead to enhanced operation of robotic devices, and in particular prosthetic devices.

The implicit motor control training system provides a novel approach to myoelectric prosthetic controls and rehabilitation and supports the use of non-intuitive, human embedded prosthetic controls. Given a myoelectric prosthetic device with N (N>0) degrees of freedom, the implicit motor control training system provides a control scheme and training system to help a user intuitively operate the prostheses using M>N residual muscles. The mapping between M filtered EMG inputs and N control outputs is an arbitrary linear transformation via a matrix of rank N. This provides proportional and simultaneous control over all N degrees of freedom. For enhanced user-friendliness, the matrix may have unit length column vectors, zero mean row vectors, and maximum angle between column vectors. The relationship between residual muscles and control outputs is arbitrary, and this disclosure describes that the training system is used to invoke intuitive control. In addition, the training system is an alternative myoelectric interface (visual or physical) with N operational degrees of freedom. The interface preferably has less physical constraints than the prosthetic device, thereby offering purer feedback to the user during interaction. The interface is designed with N operational degrees of freedom analogous to the N degrees of freedom on the prosthetic device. The interface requires the same M filtered EMG inputs as the prosthetic device, and uses the same linear mapping from inputs to outputs. The interface may depict any controllable object, real or imaginary, that is not a direct representation of the prosthetic device or amputated limb. The interface provides objective tasks with performance metrics, allowing the user to interact with the inputs required to operate the prosthetic device. The tasks may have variable attributes such as difficulty, timing, and active degrees of freedom, and the object may be configurable to keep the user engaged over time. As the user interacts with the training system, motor learning will induce the development of specific muscle synergies needed for intuitive control of the prosthetic device. When performed as part of early rehabilitation and before the prosthesis is designed, the user has an opportunity to develop these synergies such that the first interaction with the prosthesis feels intuitive and comfortable. While further interacting with the training system, these muscle synergies are refined, offering enhanced precision and functionality while interacting with the prosthetic device. The interface is designed to motivate the user to engage consistently, similar to the conceptual addictiveness of video games, but with the customized control scheme offering a full analog to controlling all active degrees of freedom on the physical prosthetic device. Thus, there is no retraining or recalibration required to maintain performance, thereby providing opportunity for enhanced acceptance and prosthetic devices designed for a more general population.

The experiment that applies the implicit motor control training system is designed to evaluate IMCTS for robust and intuitive control of robotic devices. Six healthy subjects (2 male, 4 female, aged 19-28) are evenly split into two groups, control and experimental, while learning a non-intuitive control scheme. The control group interacts directly with a 3DOF robotic application using a KUKA Light Weight Robot 4 (LWR 4) and an attached Touch Bionics iLIMB Ultra bionic hand to grasp objects. The experimental group interacts with an analogous 3DOF visual interface to implicitly learn the robot controls. Moving the robot arm in 2D is visually represented as moving a helicopter on the 2D screen, and grasping an object is visually represented as landing the helicopter onto a helipad. Both groups interact with their respective interface over two 50-minute sessions. A testing phase evaluates performance of both groups as they perform a set of tasks with the robotic device. All subjects gave informed consent of the procedures approved by the ASU IRB (Protocol: #1201007252).

### Experimental Setup

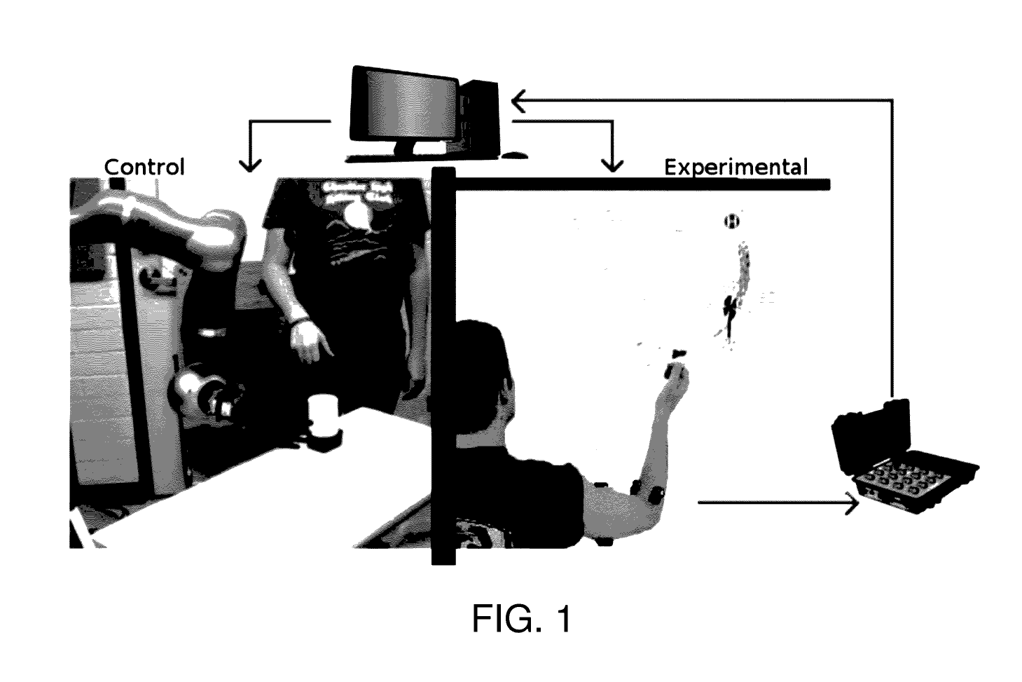

The setup for this experiment is shown in FIG. 1. Four wireless surface EMG electrodes (Delsys Trigno Wireless, Delsys Inc.) are placed on a subject's unconstrained right arm to record muscle activity from the Biceps Brachii (BB), Triceps Brachii (TB), Flexor Carpi Ulnaris (FCU), and Extensor Carpi Ulnaris (ECU). The signals are digitized at 2 kHz and sent over TCP/IP as input to a custom program using C++ and OpenGL API [12] to control either interface.

### Proportional Control

Both interfaces utilize 3 proportional control outputs corresponding to velocities of the 1) 2D planar x-axis, 2) 2D planar y-axis, 3) hand opening/closing and helicopter rising/landing. Raw EMG signals are rectified, filtered (2nd order Butterworth, cut-off 8 Hz), and normalized according to each signal's baseline e_{b} and maximal voluntary contraction e_{c} recorded at the start of each experiment:

$$e = \frac{{e}_{filt} - {e}_{b}}{{e}_{c} - {e}_{b}} .$$

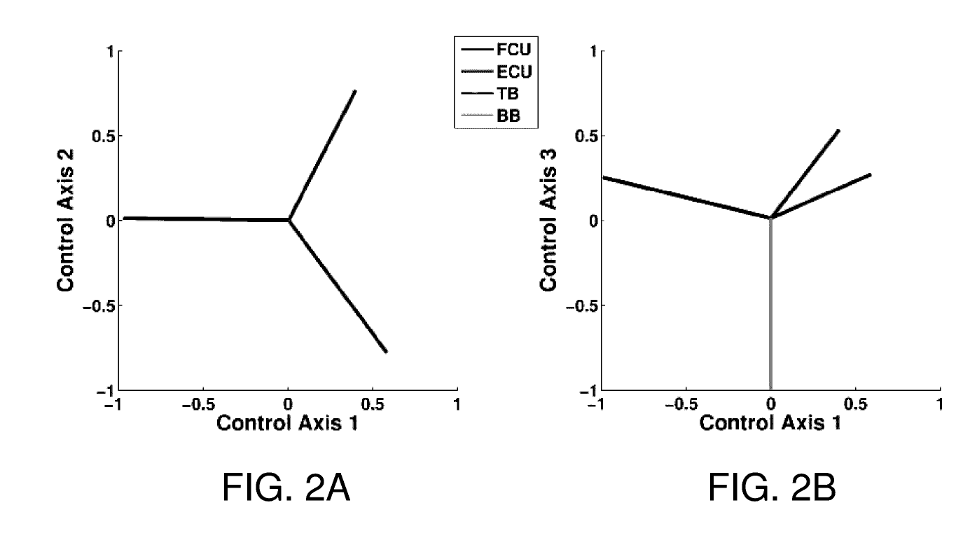

The processed signal provides a stable 4×1 input vector e of normalized EMG amplitudes which is mapped linearly to a 3×1 vector u of control outputs:

$$u = gW [ ( e - σ ) \cdot u ( e - σ ) ] , \\ W = [ \begin{matrix}- 0.9719 & 0.5775 & 0.3944 & 0.000 \\ 0.0118 & - 0.7757 & 0.7639 & 0.000 \\ 0.2361 & 0.2544 & 0.5098 & - 1.0000\end{matrix} ]$$

where o is an element-wise matrix multiplication, u(x) is the unit step function, σ=0.01 is the muscle activation threshold, and g=1.2 is the output gain. W is a random matrix optimized with respect to a cost function maximizing the angles between row vectors and subject to the following constraints (see FIG. 2): 1) One column vector is negative along the third control axis, and zero elsewhere, to disconnect grasping/landing from 2D motion. 2) All column vectors are unit length. 3) All row vectors are zero mean to prevent motion at equal co-contractions.

### Experimental Procedure

The experiment consists of both learning and testing phases over a three-week span. Subjects were initially shown example tasks with the interface, but not told how EMG maps to control outputs. The learning phase indicates performance trends as each group learns to operate the respective interface. The testing phase compares performance between groups as they both perform tasks with the robotic device.

Learning Phase: During the learning phase, subjects interact with either the robot or visual interface for 50 minutes over two separate sessions, with each session separated by one week. Within each session, subjects operate the device for two sets of 25 minutes. Within each set, subjects attempt to perform as many tasks as possible while discovering the control scheme. After each successful task, subjects rest for 7 seconds while the interface resets with a new target. At the end of the learning phase, a subject has interacted with the interface for a total of 100 minutes, 50 minutes each week.

Visual Interface: The visual interface presents a helicopter and a randomly generated path to one of 16 helipads arranged around the unit circle. The helipads are randomly arranged within each cycle of 16 tasks. The path is generated using Bezier curves with four control points, with 2000 particles distributed at random offsets along the curve. After an allotted time has passed at a given point on the path, particles turn black and can no longer be collected. A subject's score is reflected by how many particles the helicopter collects on the way to the helipad. A perfect score can be achieved by traversing the center of the path within eight seconds, thereby encouraging constant improvements in both speed and precision while learning controls. Each task is complete once the helicopter lands on the helipad.

Robot Interface: The robot interface presents the iLIMB hand which can move along a 2D plane to grasp a cylindrical object at one of 8 different locations arranged around a semicircle. The locations are randomly arranged to appear twice within each cycle of 16 tasks, and, due to the fixed hand orientation, subjects must move the hand along a specific path in order to approach and grasp the object. If the object is knocked off its location, the experimenter places it back. Each task is complete once the hand grasps the object.

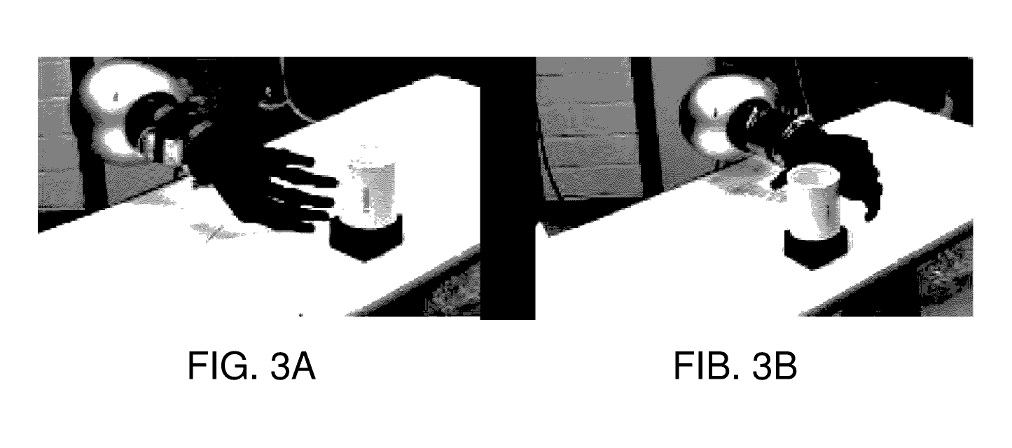

Testing Phase: The testing phase occurs a week after completion of the learning phase. Both groups control the robot interface, performing the same tasks as in the learning phase for the control group, with an additional objective of returning the object to the starting position. Moreover, after 2 cycles, or 32 tasks, the hand is rotated, as shown in FIG. 3. The changes are made to evaluate performance over generalized tasks within the same control space. The experimental group is informed that the controls require similar commands as learned in the visual interface, but are not given the exact relationship, and the control group is assured the controls are the same as the previous two weeks.

### Data Analysis

Performance is measured in the visual interface by completion time and path efficiency. Completion time is defined as the time elapsed from the start of the task until the helicopter lands on the helipad. Path efficiency is represented by the percentage of total particles collected for each trial measuring both speed and precision as a robust metric for overall control efficiency. Performance is measured for the robotic interface by completion time, defined as the time elapsed from the start of the task to grasping the object.

### Results

IMCTS is evaluated with respect to performance trends from each group in the learning phase and direct performance comparisons between the groups in the testing phase.

### Learning Phase

Due to the non-intuitive control scheme, each subject experiences a large learning curve with variable learning rates according to how efficiently the subject explores the control space. Although both interfaces are similar in terms of required inputs to complete a task, the visual interface is capable of consistently better completion times due to the lack of physical constraints such as joint velocity limits with the LWR 4, variable delays in Bluetooth communication with the iLIMB, and replacing the object if it is knocked off its location. These physical constraints slow the learning rate of the control group, as visual feedback sometimes reinforces incorrect mappings between input and outputs.

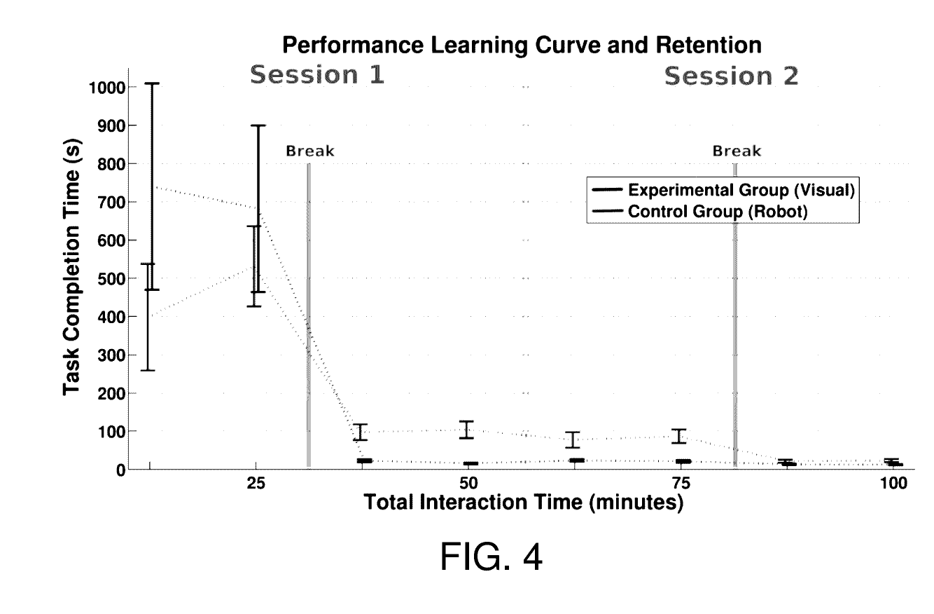

FIG. 4 displays the learning curves of both groups with average completion times as a function of total training time. Each 25 minute set of trials produces two data points, the first representing completion times over the first 12.5 minutes, and the second representing aggregated completion times over the second half of the set. The experimental group generally improved performance within each set as they refined controls. In contrast, the control group generally lowered performance between the two halves of each set. Qualitative feedback from subjects suggests that this results from tension and fatigue due to inconsistent visual feedback. This effect is reduced as subjects learn better control over time.

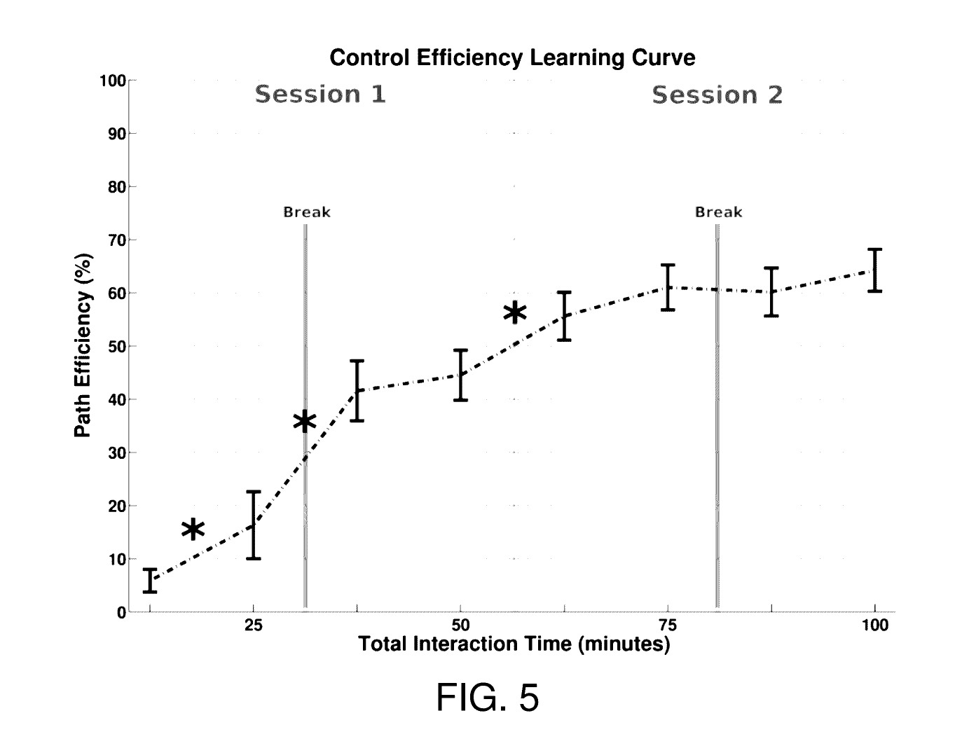

Despite having a week between sessions, both groups demonstrated performance robust to significant degradation, with the control group achieving significantly better performance between the end of session 1 and the start of session 2 (Welch's t-test, p<0.05). The experimental group traded slower performance in exchange for significantly better efficiency (Welch's t-test, p<0.05), as shown in FIG. 5. At the conclusion of the 100 minute learning phase, subjects had generally learned the mappings associating muscle activity with control outputs, but had not yet achieved consistent performance associated with fully developed muscle synergies.

### Testing Phase

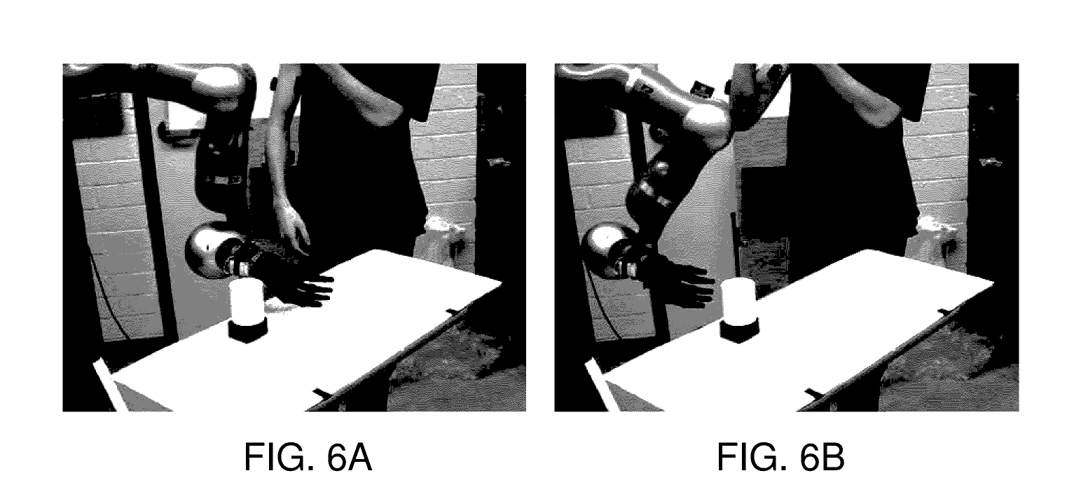

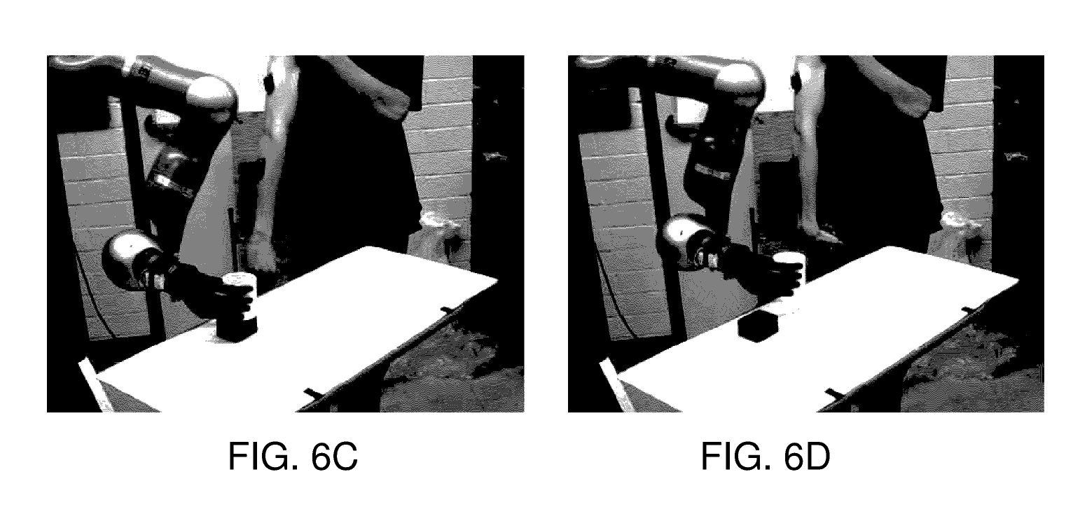

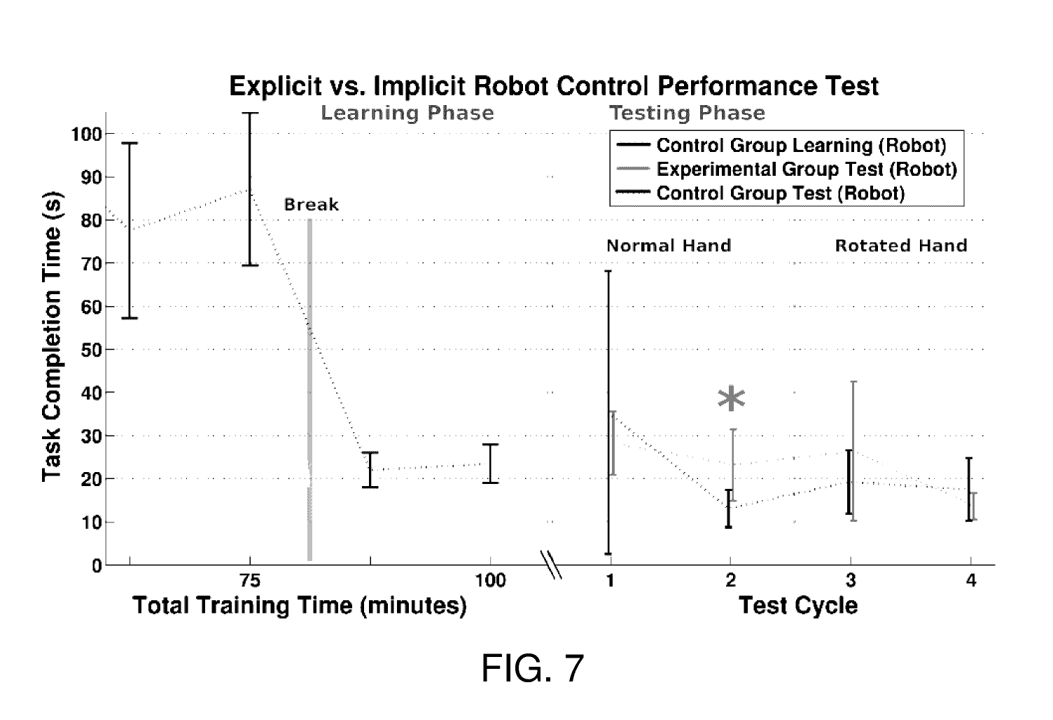

Completion times from the testing phase validate the use of IMCTS for robust robotic control. An example task sequence is shown in FIG. 6. Despite a week off and not knowing how controlling the helicopter relates to controlling the robotic hand, subjects in the experimental group are able to transfer their learning to intuitively perform the tasks comparable to the control group, with initial performance significantly better than the control group achieved after 75 minutes of total training time (Welch's t-test, p<0.05, see FIG. 7). In addition, both groups adjust to tasks with the rotated hand without a significant reduction in performance (Welch's t-test, Experimental: p=0.73, Control: p=0.15), indicating robust control of the full task space. During the fourth cycle in the test phase, the experimental group performed slightly better than the control group (Welch's t-test, p=0.17), and significantly better than the control group after 100 minutes of training (Welch's t-test, p<0.05). This, combined with the consistent learning shown in FIGS. 4 and 5, supports IMCTS as a viable tool in robotic control.

This disclosure validates the use of implicit motor control training systems to achieve intuitive and robust control of myoelectric applications. Subjects implicitly develop motor control patterns needed to control a physical robotic application through an analogous visual interface without the associated physical constraints which may hinder learning. During the learning process, subjects consistently enhance performance even after time off, corresponding to robust identification of the non-intuitive mapping function. Despite having a week off between sessions, subjects intuitively transferred their learning to efficiently control the robotic device, with performance similar to the control group, which had learned the controls by explicitly operating the robotic device for the same amount of time. These findings support the use of IMCTS to achieve practical multifunctional control of a wide range of myoelectric applications without limiting them to either intuitive mappings or anthropomorphic devices.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A method for implicit functional prosthetic device training, comprising:
connecting a user to a plurality of electromyography (EMG) sensors, each EMG sensor capable of transmitting one or more EMG signals indicative of user muscle activity;
communicably coupling the plurality of EMG sensors to a processing element capable of controlling a prosthetic device having one or more degrees of freedom;
providing an interactive task at an analogous user training interface that simulates user operation of the prosthetic device;
processing one or more EMG signals received in response to a user interaction with the interactive task and generating one or more control outputs for controlling the prosthetic device; and
receiving the one or more control outputs at the analogous user training interface and providing a real-time performance feedback.

2. The method of claim 1, wherein the real-time performance feedback comprises a real-time visual feedback indicative of user completion of the interactive task.

3. The method of claim 2, wherein the real-time performance feedback is based on one or more of the user's efficiency, accuracy, or precision in performing the interactive task.

4. The method of claim 1, wherein the one or more control outputs provide simultaneous and proportional control of each of the one or more degrees of freedom of the prosthetic device.

5. The method of claim 4, further comprising:
filtering and normalizing each of the one or more EMG signals; and
mapping the filtered and normalized EMG signals to the one or more control outputs using a pre-defined linear transformation.

6. The method of claim 5, wherein the linear transformation uses a matrix with rank equal to the number of degrees of freedom of the prosthetic device.

7. The method of claim 1, wherein the number of EMG sensors is greater than or equal to a number of residual muscles used to control the prosthetic device.

8. The method of claim 7, wherein the number of EMG sensors is greater than the number of degrees of freedom of the prosthetic device.

9. The method of claim 1, wherein the plurality of EMG sensors comprises a plurality of skin surface EMG electrodes and each of the plurality of EMG sensors is associated with a specific user muscle or muscle group.

10. The method of claim 1, wherein the user training interface can be adjusted to simulate user operation of a selected one of a plurality of different prosthetic devices.

11. An implicit prosthetic device motor control training system comprising:
a plurality of electromyography (EMG) sensors connected to a user, each of the plurality of EMG sensors capable of transmitting one or more EMG signals indicative of user muscle activity;
a robotic device having one or more degrees of freedom;
a processing element communicably coupled to the plurality of EMG sensors, the processing element capable of controlling the robotic device by processing one or more EMG signals to generate one or more control outputs; and
an analogous user training interface designed to simulate user operation of the prosthetic device in an interactive task by receiving the one or more control outputs and generating real-time performance feedback indicative of user completion of the interactive task.

12. The system of claim 11, wherein the real-time performance feedback comprises a real-time visual feedback based on one or more of the user's efficiency, accuracy, or precision in performing the interactive task.

13. The system of claim 11, wherein the one or more control outputs provide simultaneous and proportional control of each of the one or more degrees of freedom.

14. The system of claim 13, wherein the processing element is further capable of:
filtering and normalizing each of the one or more EMG signals; and
mapping the filtered and normalized EMG signals to the one or more control outputs using a pre-defined linear transformation.

15. The system of claim 14, wherein the linear transformation uses a matrix with rank equal to the number of degrees of freedom of the robotic device.

16. The system of claim 11, wherein the number of EMG sensors is greater than or equal to a number of residual muscles used to control the robotic device.

17. The system of claim 16, wherein the robotic device comprises a prosthetic device.

18. The system of claim 11, wherein the plurality of EMG sensors comprises skin surface EMG electrodes and each of the plurality of EMG sensors is associated with a specific user muscle or muscle group.

19. The system of claim 11, wherein the user training interface can be adjusted to simulate user operation of a selected one of a plurality of different robotic devices.

20. The system of claim 11, wherein the robotic device is optional, and the plurality of EMG sensors record user muscle activity from muscles requiring rehabilitation via implicit training with the analogous user training interface.
